(19) United States
(12) Patent Application Publication
(10) Pub. No.: US 2015/0241505 A1
(43) Pub. Date: Aug. 27, 2015

(54) System And Method For Measuring Contact Impedance Of An Electrode

(71) Applicant: Draeger Medical Systems, Inc., Andocer, MA (US)

(72) Inventors: Daniel Freeman, Somerville, MA (US); Clifford Mark Risher-Kelly, Wells, ME (US); David C. Maurer, Stoneham, MA (US)

(73) Assignee: Draeger Medical Systems, Inc., Andover, MA (US)

(21) Appl. No.: 14/418,400

(22) PCT Filed: Aug. 1, 2012
(86) PCT No.: PCT/US2012/049121

§ 371 Date: Jan. 29, 2015

(51) Int. Cl. G01R 31/04; A61B 5/044; A61B 5/04; A61B 5/0408; A61B 5/00

(52) U.S. Cl. CPC G01R 31/04; A61B 5/0408; A61B 5/7221; A61B 5/6843; A61B 5/04012; A61B 5/746; A61B 5/7246; A61B 5/044; A61B 5/0006; A61B 2562/0214

(57) ABSTRACT

An apparatus and method that determines a quality of a connection of an electrode to a patient is provided. The apparatus includes at least three electrodes selectively connected to a patient for sensing an electro-physiological signal representing a patient parameter. A current source is connected to each of the at least three electrodes, the current source able to apply both a positive current and a negative current. A control processor is connected to the current source and the at least three electrodes. The control processor identifies a number of unique electrode pairs of the at least three electrodes and controls the current source to simultaneously apply a positive current to one electrode and a negative current to an other electrode of each identified electrode pair to determine a connection quality for at least one of the at least three electrodes.

### FIELD OF THE INVENTION

This invention concerns a system and method for patient monitoring devices and, more specifically, for measuring the contact impedance of an electrode to determine a connection quality associated with the electrode.

### BACKGROUND OF THE INVENTION

In the course of providing healthcare to patients, it is necessary to monitor vital statistics and other patient parameters. Different types of patient monitoring devices are able to monitor the physiological state of the patient via at least one electrode that is coupled to the skin of the patient at various locations on the body. For example, the electrical activity of the heart is routinely monitored in clinical environments using an electrocardiogram (ECG) monitor. The ECG monitor is connected to the patient by a plurality of electrodes that monitor the electrical impulses of the patient's heart. In order for the ECG monitor to effectively record the electrical impulses of the patient, electrodes extending therefrom conventionally include a conductive gel that is embedded in an adhesive pad used to secure the electrode to the body of a patient. Wires from the monitor are selectively connected to the electrode in order to communicate voltages detected to the ECG monitoring device to provide a healthcare practitioner with data regarding the patient's heart function.

It is well known that the quality of the recorded signal depends on the electrical resistance between the electrode and the patient's body. The resistance at the electrode-patient interface is known as contact impedance. Therefore, it is desirable to measure the contact impedance at various times while the patient is being monitored thereby ensuring that the signal being monitored is of a sufficient quality. One approach for measuring contact impedance is to use pull-up/pull-down resistors where each electrode is connected to a resistor (generally tens of megaohms) in series with a voltage source. This will cause the electrode voltage to be drawn near the applied voltage level when the contact impedance increases to the tens of megaohms range. This indicates the presence of a poor connection and suggests the signal being sensed is of sub-optimal quality. Another approach to measuring contact impedance is to apply a current to a given electrode which returns to ground through the other connected electrode. This results in a voltage drop across the electrode to which the current was applied and a corresponding voltage drop across a parallel combination of all other electrodes. By repeating this measurement for each of N total electrodes, a set of N nonlinear equations and N-unknowns can be derived where N is equal to the number of electrodes connected to the system. However, certain drawbacks are associated with these and other approaches to measuring contact impedance including providing a less reliable measurement of signal quality and increased computational time and complexity needed by the system to generate this measurement. A system according to invention principles addresses deficiencies of known systems.

### SUMMARY OF THE INVENTION

In one embodiment, an apparatus that determines a quality of a connection of an electrode to a patient is provided. The apparatus includes at least three electrodes selectively connected to a patient for sensing an electrophysiological signal representing a patient parameter. A current source is connected to each of the at least three electrodes, the current source able to apply both a positive current and a negative current. A control processor is connected to the current source and the at least three electrodes. The control processor identifies a number of unique electrode pairs of the at least three electrodes and controls the current source to simultaneously apply a positive current to one electrode and a negative current to an other electrode of each identified electrode pair to determine a connection quality for at least one of the at least three electrodes.

In another embodiment, an ECG monitoring apparatus that determines a connection quality of an electrode connected to a patient. The ECG monitor includes a plurality of electrodes coupled to a patient, each of the plurality of electrodes sensing electrical impulses representing at least one patient parameter from the patient. A current source is selectively connectable to the plurality of electrodes that selectively applies one of a positive current and negative current to any of the plurality of electrodes. A control processor is connected to the current source and the plurality of electrodes. The control processor identifies a number of unique electrode pairs from the plurality of electrodes and, for each identified electrode pair, controls the current source to apply a positive current one of the electrodes and a negative current to the other of the electrodes in the electrode pair to generate linear equations representing the voltage difference for the electrode pair and determines connection quality data for respective ones of the plurality of electrodes.

In another embodiment, a method of determining a connection quality of an electrode connected to a patient is provided. The method includes the activities of providing at least three electrodes that sense an electrophysiological signal representing a patient parameter to a control processor. A current source able to apply both a positive current and a negative current is connected to each of the at least three electrodes. A number of unique electrode pairs of the at least three electrodes is identified and the current source is controlled to simultaneously apply a positive current to one electrode and a negative current to an other electrode of each identified electrode pair. A connection quality is determined for at least one of the at least three electrodes.

### BRIEF DESCRIPTION OF THE DRAWINGS

FIG. 1 is an exemplary block diagram of the system for measuring contact impedance of an electrode according to invention principles;

FIG. 1A illustrates the use of the neutral electrode to reduce the level of common mode noise recorded by ECG monitors. The average voltage of the three primary electrodes is input to an inverting amplifier whose output connects to the neutral electrode.

FIG. 2 depicts an exemplary circuit diagram of an electrode connected to a patient for use in the system for measuring contact impedance according to invention principles;

FIG. 3 is an exemplary circuit diagram of electrodes connected to a patient for use in the system for measuring contact impedance according to invention principles;

FIG. 4 is an exemplary circuit diagram of electrodes connected to a patient for use in the system for measuring contact impedance according to invention principles;

FIG. 5 is an exemplary circuit diagram of electrodes connected to a patient for use in the system for measuring contact impedance according to invention principles;

FIG. 5A is an exemplary circuit diagram of electrodes connected to a patient for use in the system for measuring contact impedance according to invention principles;

FIG. 6 is a table representing the voltages of each electrode pair connected to a patient for use in measuring contact impedance according to invention principles;

FIG. 7 is a flow diagram detailing the operation of the system for measuring contact impedance according to invention principles;

FIG. 8 is a flow diagram detailing the operation of the system for measuring contact impedance according to invention principles; and

FIG. 9 is a flow diagram detailing the operation of the system for measuring contact impedance according to invention principles.

### DETAILED DESCRIPTION

The system for measuring contact impedance (hereinafter “system”) automatically measures, calculates and quantifies the quality of a connection between the electrode and the patient. The connection quality is determined by measuring impedance at the interface between an electrode connected to the patient and the skin of the patient. This is known as the contact impedance and the system advantageously measures and determines the contact impedance for each electrode during the course of patient monitoring. By measuring the contact impedance during patient monitoring, healthcare practitioners may be notified in real time of a condition representative of a degrading connection of one or more electrodes connected to the patient. This enables a healthcare practitioner to remedy a situation that would otherwise lead to a signal sensed by the electrode having a less than desirable signal quality causing data that does not accurate represent a current condition of the patient to be generated. Connection quality data for a particular electrode is automatically determined using two electrodes from a set of at least three electrodes that are connected to the patient being monitored. Each electrode includes a first current source having a first polarity and a second current source having the opposite polarity connected thereto. The magnitude of the current from each of the first and second current source is equal to one another. The system automatically measures and determines a connection quality of the electrodes connected to a patient by selectively applying the first current source having a predetermined magnitude to one of the three electrodes and simultaneously applying the second current source having the predetermined magnitude to another one of the three electrodes. The system sequentially applies the first and second current source to each combination of electrode pairs for the three electrodes. In this manner, the system advantageously measures a voltage differential between each of the electrodes and, because the current level is of a predetermined value, the system automatically generates, for each electrode pair, a linear equation wherein the voltage differential between two electrodes is equal to the sum of the contact impedances of a first and second electrode times the current level. However, the respective impedances of the first and second electrodes are unknown until the system generates an equation representing each electrode pair. Thereafter, the three generated linear equations representing each electrode pair (e.g. Electrode 1 and 2, Electrode 2 and 3, and Electrode 1 and 3) may advantageously be mathematically manipulated to resolve the respective contact impedances for each electrode. In response to determining the contact impedances for respective electrodes, the contact impedance values are compared to a threshold contact impedance to determine if the signal being sensed by the respective electrode is of a sufficient quality whereby a lower impedance correlates to a stronger connection at the electrode/patient interface. By automatically and simultaneously applying two opposite polarity current sources to two different electrodes the system may determine the signal quality in a reduced time thereby reducing the impact on patient monitoring. The reduction in time needed to determine the signal quality using the present system is achieved due to the reduced computational processing requirements of generating and resolving three linear equations each representing a respective electrode pair. The system provides a further advantage by identifying the connection quality of electrodes to select a lead combination that provides highest quality ECG data. Moreover, the connection quality data provided by the system enables a user to determine what combination of ECG leads may be used at a given time.

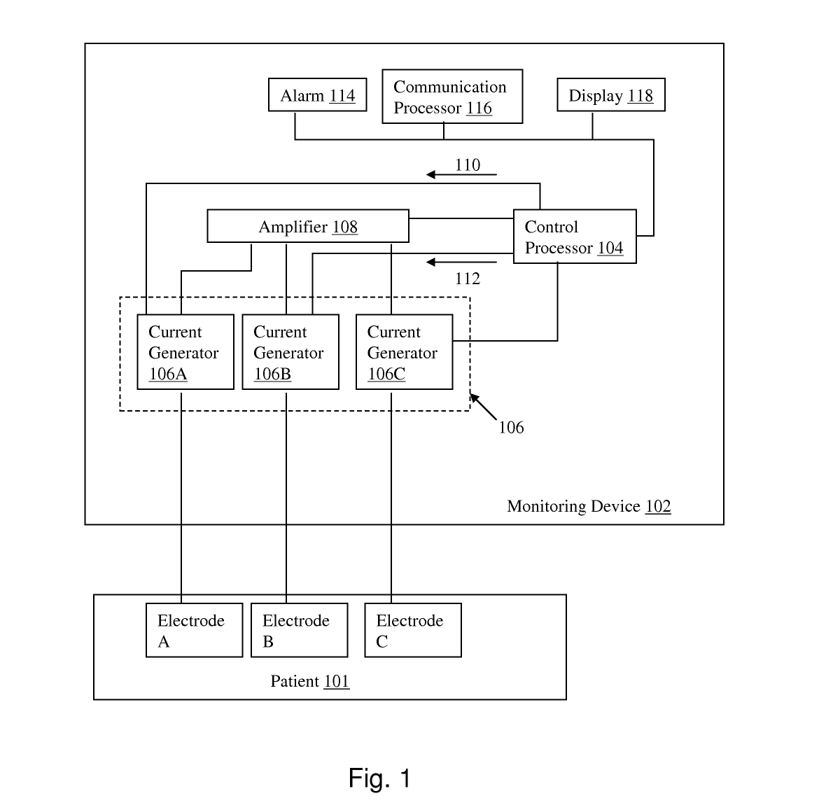

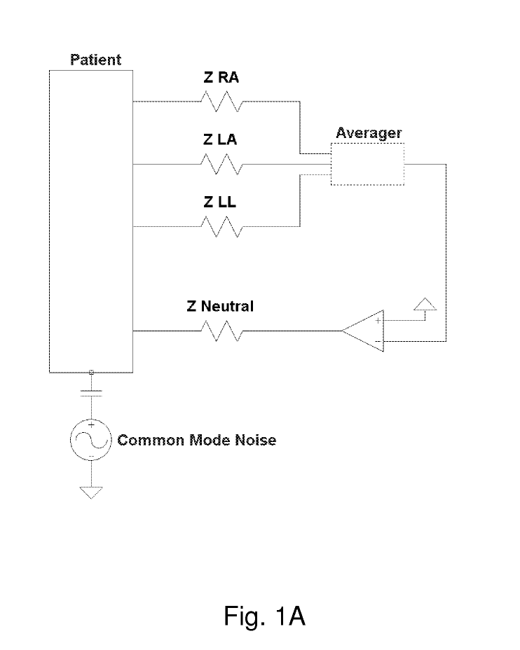

FIG. 1 is a block diagram of an exemplary patient monitoring device 102 that selectively monitors electrical impulses from a patient via a plurality of electrodes A-C that are connected to predetermined locations on the patient using a conductive gel and an adhesive. While electrodes A-C are shown herein, it should be appreciated that any number of electrodes may be used to monitor the electrical impulses of the patient and the number of electrodes employed depends on the type of data being monitored by the monitoring device 102. In one embodiment, the patient monitoring device is an ECG monitor and the plurality of electrodes may include electrodes that are attached to the patient's limbs and chest. One skilled in the art understands that the electrodes are commonly positioned on the right arm (RA), left arm (LA), right leg (RL), left leg (LL), and in some cases there are several electrodes placed on the chest. Of these electrodes, RA, LA and LL are generally referred to as “primary electrodes”, the electrodes on the chest are referred to as “V-leads”, and RL is usually referred to as the “neutral electrode”, although in practice, any electrode can be designated as the neutral. We use the term “secondary electrodes” to refer collectively to the V-leads and neutral electrode. The primary electrodes RA, LA and RL are coupled to an averager which automatically averages the voltages of the primary electrodes in order to generate a reference voltage known as the Wilson Point. An illustration of the three primary electrodes and the neutral electrode is shown in FIG. 1A, where Z_RA, Z_LA, Z_LL, and Z_Neutral represent the electrode impedances of the RA, LA, LL, and neutral electrodes, respectively. The reference voltage generated by the averager may be used as will be discussed below to determine the impedances for any the V-leads. There is significant noise that is introduced to the body through capacitive coupling to various sources of electric fields (e.g. power line noise). This noise is referred to as common mode noise and it can obscure the ECG signal. Therefore, in order to reduce such noise, most ECG monitors will take the average voltage of the three primary electrodes and then input this signal to an inverting amplifier. The output of this amplifier is connected to the neutral electrode. By inverting the signal that is common to all electrodes and injecting it back into the body, the noise levels experience by the ECG monitor is dramatically reduced. Additionally, the V-leads are known to include electrodes V₁-V₆ positioned at predetermined locations on the chest of the patient in a known manner. The manner in which the monitoring device monitors the electrical impulses to generate and output ECG waveforms is known and is not germane to the present invention and will not be discussed further.

Referring back to FIG. 1, the monitoring device 102 includes a control processor 104 that includes control logic to control the operation of the monitoring device 102. The control logic includes algorithms for monitoring the electrical impulses of a patient to produce patient parameter data (e.g. ECG waveform). The system further includes a plurality of current generators 106A-106C associated with a respective electrode. The current generators 106A-106C are collectively referred to herein using reference numeral 106 and one should appreciate that any function described with respect to element 106 may be accomplished by any of the respective current generators 106A-106C. Each current generator 106 is electrically coupled to the control processor 104 and is under selective control thereby as will be discussed below. While FIG. 1 shows each respective current generator 106A- 106C being directly connected to the control processor 104, one skilled in the art of electric circuit design would appreciate that there are other equivalent methods and circuit designs that would enable the control processor 104 to be selectively coupled to the respective current generator 106 and achieve the same objective. The current generator 106 is able to selectively apply one of a first current having a first polarity or a second current having a second opposite polarity to a respective electrode at a given time. For example, the current generator 106 includes a positive current source 107 and a negative current source 109 (e.g. a current drain) that is connected to the negative power supply (-AVDD). As shown herein, this embodiment includes a first current generator 106A coupled to the first electrode A, a second current generator 106B coupled to the second electrode B and a third current generator 106C coupled to the third electrode C. As described above with respect to the number of electrodes, the present system may include a number of current generators 106 equal to the number of electrodes connected to the patient monitoring device 102. In another embodiment, a reduced number of current generators may be used with the system and coupled to different electrodes via various switching arrangements thereby minimizing the electrical complexity of the monitoring device 102.

The control processor 104 selectively controls two of the respective current generators 106 at a given time to automatically apply a current of the same magnitude but of opposite polarities to a respective electrode pair. By automatically and simultaneously applying opposite polarity current to two different electrodes, the system advantageously defines the precise path of current flow at a particular time. By defining the precise path of current flow, a voltage differential across the two electrodes to which the current is being simultaneously applied may be measured. An amplifier 108 is electrically coupled between the control processor 104 and each respective electrode A-C. The amplifier 108 selectively measures and compares a voltage difference between two respective electrodes connected to the patient 101. Voltage differential data of a respective electrode pair may be automatically provided to the control processor 104 for use in calculating contact impedance associated with each electrode of the electrode pair through which current is flowing.

In operation, the control processor 104 selectively identifies a number of electrode pairs based on the number of electrodes connected to the patient monitoring device 101. The control processor 104 may identify the number of electrodes in any known manner such as sensing if a voltage at a particular connector is present or by querying configuration information entered by a healthcare practitioner that identifies a number and configuration of electrodes at a given time. Upon identifying a number of electrode pairs, the control processor 104 determines a number of linear equations representing the contact impedances of the electrode pair that are needed in order to determine the contact impedance for each electrode of the electrode pair. Exemplary operation will be described with respect to an electrode pair including Electrodes A, B and C. The control processor 104 generates and provides a first control signal 110 to current generator 106A connected to electrode A. The first control signal 110 may include information identifying a polarity of the current to be applied to the electrode and a magnitude of the current being applied to the electrode. In another embodiment, the first control signal 110 may also include a duration for which the current will be applied to the electrode. While the content of the control signal is described with respect to the first control signal 110, one skilled in the art will understand that each control signal generated by control processor 104 may include the same type of data but having different values (e.g. different polarities). Simultaneously with the generation of the first control signal 110, the control processor 104 generates and provides a second control signal 112 to current generator 106B coupled to Electrode B. The second control signal 112 causes the current generator 106B to apply a current to Electrode B having a same magnitude and a polarity opposite to the current being applied to Electrode A. In response to the simultaneous application of currents of the same magnitude and opposing polarities to electrodes A and B, the control processor 104 causes the amplifier 108 to automatically measure a voltage difference between Electrode A and B. For each electrode pair identified, the control processor 104 generates an equation for use in determining the contact impedance for each electrode in the set of electrodes. A first equation is a linear equation whereby the measured voltage difference of Electrode A and B is equal to the product of the current and the sum of the impedances of Electrodes A and B. However, as the individual impedances of Electrodes A and B are unknown, the control processor 104 automatically repeats the above operation for each other respective electrode pair (e.g. Electrode B and C; and Electrode A and C) to generate respective second and third linear equations. In response to generating a number of linear equations equal to the number of identified electrode pairs, the control processor 104 automatically solves calculates the contact impedance for each of the electrodes using the three equations to solve for respective values representing the contact impedance of each electrode A-C. This calculation is possible because the voltage difference and current applied to each electrode pair is known. An important aspect of this method is that the time required to calculate the contact impedance for each electrode is reduced compared to previous methods. For example, consider the method in which a single current source is applied sequentially to all electrodes while all other electrodes are tied to ground. This method produces a set of N nonlinear equations, where N equals the number of electrodes. These nonlinear equations cannot be solved explicitly for impedance and therefore must use more computationally intensive methods that require a longer time to solve compared to the new method described here.

The resulting contact electrode impedance for each electrode is compared to a threshold contact impedance to produce connection quality data for the selected electrode. If the resulting electrode impedance is below the threshold level, the connection quality is determined to be good. If the resulting impedance is equal to or greater than the threshold level, the connection quality is determined to be poor. For example, electrode impedance may range between 50 kΩs and tens of mega ohms, whereby a lower impedance indicates a higher quality of the connection at the patient/electrode interface. In one embodiment, there may be a scale of connection quality data identifiers that, based on the resulting impedance, provide a user with a greater level of information about the connection quality beyond “good” and “poor”.

The monitoring device 102 further includes an alarm 114, a communication processor 116 and a display 118 each connected to the control processor 104. Upon determining connection quality data for each electrode A-C, the control processor 104 may provide the connection quality data for output to a user. In one embodiment, should the connection quality data determined for the selected electrode indicate the connection is poor, the control processor 104 may automatically cause an alarm 114 to be issued. The alarm may be any of a tactile, audio or visual alarm (or any combination thereof) that notifies a healthcare practitioner that the connection of at least one electrode is poor. The healthcare practitioner is then alerted to rectify the connection to the patient to ensure high quality patient monitoring. In another embodiment, the connection quality data for each electrode can be collected and provided to a communication processor 116 for communicating the connection quality data to a remote system. The communication processor 116 may be connected to a communication network (wired or wireless) and transmit connection quality data to a patient management system for inclusion in a patient record. The communication processor 116 may employ known communication protocols to communicate over cellular networks, a local area network and/or wide area networks. In a further embodiment, connection quality data may be used to modify a display image on a display device 118. For example, the control processor 104 may generate a connection quality indicator to be associated with each electrode and display the connection quality indicator on the display 118. In the event the connection quality is determined to be good, the connection quality indicator may be displayed in a first format or style. If the connection quality is ever determined to be poor, the control processor 104 may cause the connection quality indicator to change to a different format or style that notifies a user that the connection quality is poor. The manner in which the connection quality data may be used is described for purposes of example only and the connection quality data may be used for any purpose to provide patient care.

In another embodiment, the patient monitoring device may be an Electroencephalograph monitor (EEG) that senses the electrical activity along the scalp to measure voltage fluctuations resulting from ionic current flows within the neurons of the brain. In this embodiment, the principles described above may be applied in a similar manner whereby the connection quality of individual electrodes connected to the patient's scalp may be determined. However, the current applied to the electrode being measured in the case of an EEG may be an AC current as opposed to a DC current.

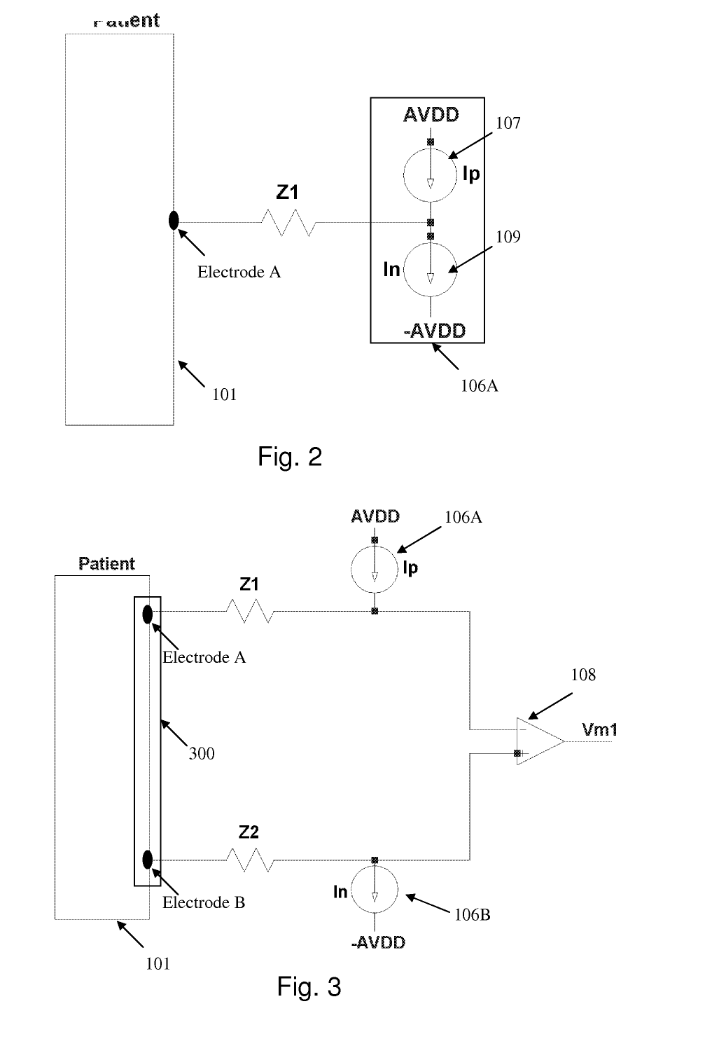

An exemplary embodiment of the contact impedance measurement system described above in FIG. 1 will now be described in further detail in FIGS. 2-5. FIG. 2 is an exemplary circuit diagram of a respective one of a plurality of electrodes connected to the patient 101. As shown herein Electrode A is releasably secured to the patient 101 in a known manner and current generator 106A is coupled to Electrode A enabling either a positive or negative current to be applied to Electrode A at a given time. Contact impedance of Electrode A is represented as resistance Z1. Thus, the objective is to automatically measure contact impedance Z1 for Electrode A to determine connection quality data for Electrode A. While FIG. 2 only depicts Electrode A, one skilled in the art will appreciate that this Figure is representative of each electrode in the set of electrodes connected to patient 101. The current generator 106 includes a dual source that can be selectively turned on/off. The first current source may apply a positive current to the electrode (Iₚ), and the second current source may apply a negative current to the electrode (Iₙ). The first current source is connected to the positive power supply (AVDD) while the second current source is connected to the negative power supply (−AVDD). The current generator 106A is selectively controlled to apply one of the first current source (positive) or second current source (negative) at a given time depending on the measurement being taken at a given time as will be discussed hereinafter with respect to FIGS. 3-5. The magnitude of the currents applied to Electrode A and any one other electrode are equal (i.e. |Iₚ|=|Iₙ=I).

Referring now to FIG. 3, an exemplary circuit diagram that illustrates two electrodes selected from a set of electrodes is shown. A representative electrode pair 300 is shown in FIG. 3. The electrode pair 300 includes Electrode A and Electrode B which are each connected to inputs of an amplifier 108. Electrodes A and B each include contact impedance Z1 and Z2, respectively, associated therewith. The system advantageously calculates the contact impedance values to determine connection quality data for each of Electrodes A and B.

The current generator 106A and current generator 106B, respectively, are responsive to control signals generated by the control processor (104 in FIG. 1). The current generator 106A is caused to apply a positive current to Electrode A while current generator 106B is simultaneously controlled to apply a negative current to Electrode B. Thus, a current path is formed through which current flows from current generator 106A through Electrode A and the patient 101 and back through Electrode B towards the current drain (negative current source) in current generator 106B. A voltage differential (Vₘ) is measured by amplifier 108 and voltage differential data for electrode pair 300 is used, in conjunction with voltage differential data associated with the other pairs of electrodes, in determining contact impedance values for Electrodes A and B.

In operation, the system advantageously measures the contact impedances Z1 and Z2. Electrode A having contact impedance Z1 is connected to a positive current source, which is tied to the power supply voltage (AVDD). Electrode B having contact impedance Z2 is connected to a negative current source, which is tied to the negative power supply (−AVDD). The differential voltage between the electrodes in the first electrode pair 300, Vₘ₁, is amplified and recorded. In order to derive the equations for use in calculating values of contact impedances Z1 and Z2, a positive current is applied to electrode Z1 while a negative current is applied simultaneously to electrode Z2. This will result in the Equation 1 below:

Vₘ₁=I (Z1+Z2) (1)

where I is known, Vₘ is measured, and Z1+Z2 are unknown.

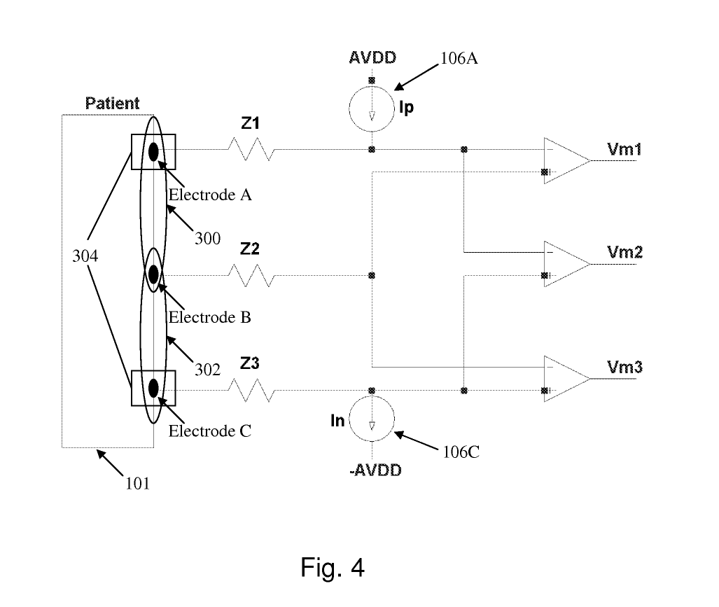

The presence of two unknown variables in a single equation prevents the system from determining the values of Z1 and Z2. Therefore, we will derive two additional equations by introducing a third electrode, Electrode C having a third contact impedance Z3, by applying pairwise stimulation between Electrode A and Electrode C, as well as Electrode B and Electrode C. The system measures a voltage differential for each of a second and third electrode pairs and determines second and third linear equations for use in determining the contact impedances associated with each of the three electrodes in the set of electrodes. By deriving three linear equations having three unknowns, the system is able to rapidly solve for each of the three unknowns rapidly. This is illustrated in FIG. 4 which depicts the system having the third electrode, Electrode C, having a contact impedance Z3 associated therewith. For simplicity, only two current sources are shown, but in practice a positive and negative current source or dual current source is attached to each electrode and is able to apply pairwise stimulation to each electrode (see FIG. 1). By applying a positive current to Z1 and negative current to Z3, and then repeating for electrodes Z2 and Z3, and including Equation 1 above, we get the following equations used to determine contact impedances Z1-Z3:

Vₘ₁=I(Z1+Z2) (1)

Vₘ₂=I(Z1+Z3) (2)

Vₘ₃=I(Z2+Z3) (3)

The values of Vₘ₁, Vₘ₂ and Vₘ₃ are stored along with the first equation represented by Equation 1, the second equation represented by Equation 2 and the third equation represented by Equation 3. As these equations are linear with respect to variables Z1-Z3, the control processor 104 in FIG. 1 can determine values for Z1, Z2, and Z3 in terms of known quantities. These resulting values for contact impedances Z1-Z3 are shown in Equations 4-6, respectively:

Z1=0.5/1*(Vₘ₁+Vₘ₂−Vₘ₃) (4)

Z2=0.5/I*(Vₘ₁+Vₘ₃−Vₘ₂) (5)

Z3=0.5/I*(Vₘ₂+Vₘ₃−Vₘ₁) (6)

The simultaneous application of two current sources of opposing polarities to two different electrodes of an electrode pair enables the values in Equations 4-6 to be readily determined near instantaneously and using a minimal amount of processing power. If only a single current source were applied, these equations would no longer hold. For example, if a positive current source was applied to Z1 without applying negative current to Z3, then the current flowing through Z1 would return through the neutral electrode and not through Z3 because electrode Z3 is connected to a high input impedance amplifier. Even if electrode Z3 were tied to ground, current injected through Z1 would return through both Z3 and the neutral electrode. Because the current through Z3 is not known in this case, the above equations would not hold. By introducing two currents sources (one positive and one negative), the system advantageously defines the path of current through any two electrodes regardless of the presence of additional electrodes.

In response to determining contact impedance values Z1-Z3 using equations 4-6, these contact impedance values Z1-Z3 are compared to threshold contact impedance values to determine connection quality data for the particular electrode connected to the patient 101.

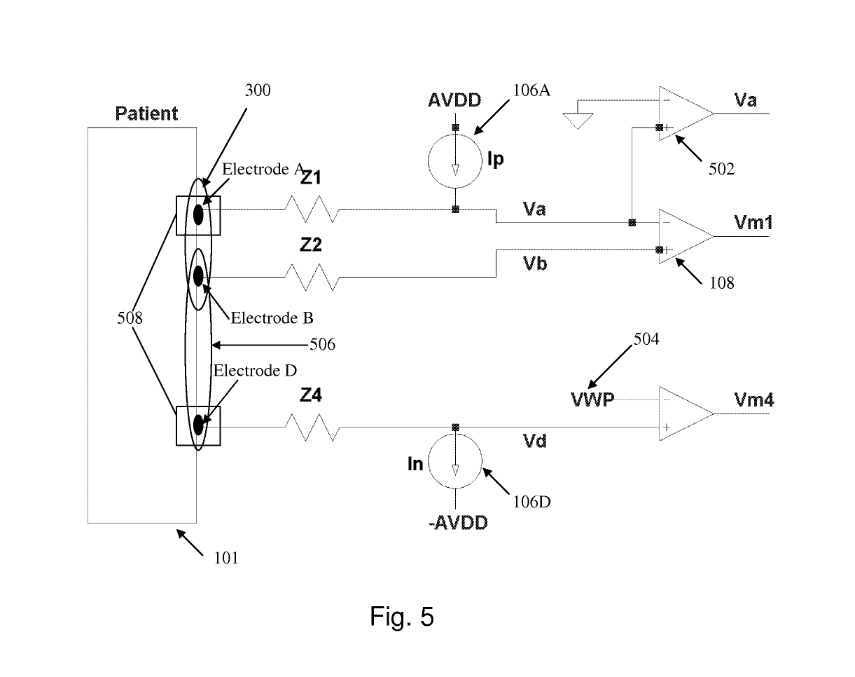

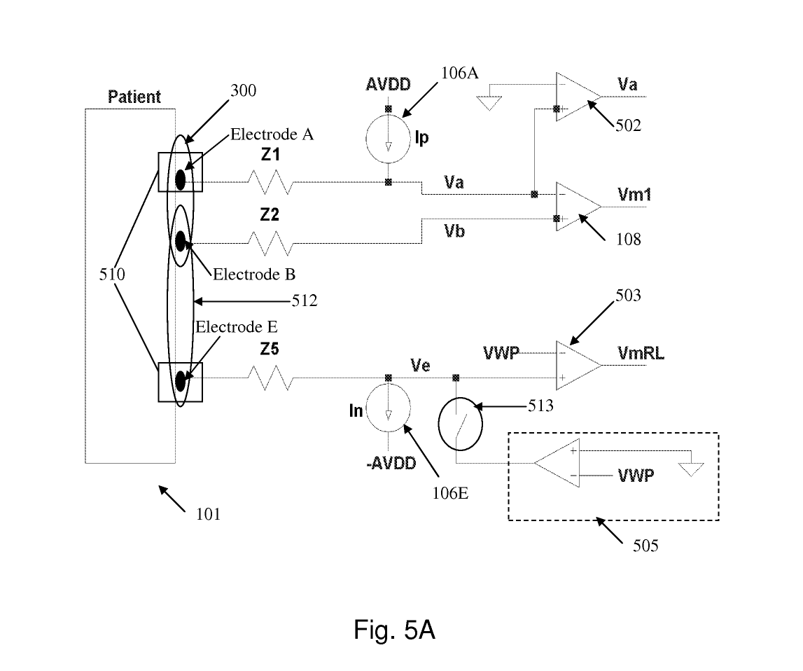

In the 3-electrode patient monitoring systems (e.g a 3-lead ECG monitoring system), the amplifiers measure and record the differential voltage between electrodes as opposed to measuring the individual electrode voltages. This is described above in FIG. 3. However, in patient monitoring systems having more than 3 electrodes (e.g. 12-lead ECG), there may be a certain number of electrodes whose voltage is measured relative to a reference voltage. In one embodiment, the reference voltage may be the Wilson point (V_{WP}) which is defined as the average voltage of the primary leads. FIG. 5 is a circuit diagram illustrating how contact impedance values associated with V-leads may be calculated.

FIG. 5 illustrates how the contact impedance Z4 of a fourth electrode, Electrode D, which represents a secondary electrode, is determined. In one embodiment, Electrode D is V-lead in an ECG monitoring system. To measure the contact impedance for Electrode D, the system measures the electrical voltage on each electrode connected to the patient. For purposes of example only, the electrodes shown in FIG. 5 are Electrode A, Electrode B and Electrode D. However, one skilled in the art will understand that the system includes similar circuitry for any number of other primary electrodes (e.g. Electrode C in FIG. 3) or any number of other secondary electrodes (not shown).

In FIG. 5, Electrode A is coupled to patient 101 and further connected to a negative input of amplifier 108 as discussed above. The description herein of electrodes being connected to either positive or negative inputs of respective amplifiers is for purposes of example only. Thus, if a first electrode is described as being connected to a positive input and a second electrode is described as being connected to a negative input, one skilled in the art would understand that the connections could readily be reversed and the system would yield the same outcome. Electrode A is also connected to a second amplifier 502 which measures the actual voltage on Electrode A represented as Va. Electrode B is coupled to patient 101 and is also connected to a positive input of amplifier 108 as discussed above to measure a voltage differential Vₘ₁ associated with electrode pair 300. The voltage on Electrode B is represented as Vb. While not shown, one skilled in the art will appreciate that a further amplifier may be connected on Electrode B similarly to second amplifier 502 on Electrode A. This further amplifier may measure and record the voltage Vb on Electrode B. Electrode D is a secondary electrode having contact impedance Z4 associated therewith and includes a voltage Vd present thereon. Electrode D further includes a current generator 106D that is able to apply either the first positive current source or the second negative current source in response to a control signal generated by a control processor (104 in FIG. 1). The voltage differential data Vₘ₄ for Electrode D measures the difference between the voltage Vd on Electrode D and a reference voltage 504. In one embodiment, the reference voltage is the Wilson Point

In operation, voltages Va, Vb, and Vd represent the electrode voltages of electrodes A, B and D, respectively. In order to measure the value of the contact impedance Z4, a further electrode pair 508 comprising Electrode A and Electrode D are used. In this manner, the current generator 106A is controlled to apply a positive current to Electrode A while simultaneously applying a negative current by current generator 106D to Electrode D. This causes current to flow through Electrode A and return through electrode D. The resulting voltage from the applied current is represented in Equation 7 as:

Va−Vd=I(Z1+Z4) (7)

The voltage Va is amplified relative to ground (gain=1) and recorded. The voltage Vd can be obtained by adding the differential voltage Vm4 to the reference voltage (V_{WP}) as shown in Equation 8:

Vd=Vm4+VWP (8)

The system repeats this process between Electrode B and Electrode D forming electrode pair 506. A similar measurement can be made between Electrode B and Electrode D where positive current is applied to Electrode B and negative current is applied to Electrode D. By repeating the above application with electrode pair 506, the values of impedances Z1, Z2, and Z4 can then be derived by modifying Equation 8 above as Equation 9 shown below and using Equation 9 in conjunction with equations listed in Equations 10 and 11 as follows:

Va−Vd1=I(Z1+Z4) (9)

Vb−Vd2=I(Z2+Z4) (10)

Vm1=I(Z1+Z2) (11)

Where Vd1 is Vd (from Eq. 8) in the case that current is injected through electrodes A and D, while Vd2 is Vd (from Eq 8) in the case that current is injected through electrodes B and D, Vm1 is measured when current is injected through electrodes A and B, Va is measured when current is injected through electrodes A and D, and Vb is valid when current is injected through electrodes B and D. While Vb is not directly measured, it can be obtained easily either by amplifying and recording Vb directly, or using the differential voltages Vm1, Vm2, and Vm3 in combination with the electrode voltage Va. Once Vb is known, the values of Z1, Z2, and Z4 can be calculated using the same manner as discussed above with respect to Equations 4-6, where Z3 would be replaced with Z4 in the embodiment shown in FIG. 5. Thus, the values of Z1, Z2 and Z4 are shown in Equations 12-14, respectively:

Z1=0.5/I*(Vₘ₁+Va−Vd1−Vb+Vd2) (12)

Z2=0.5/I*(Vₘ₁+Vb−Vd2−Va+Vd1) (13)

Z4=0.5/I*(Va+Vd1+Vb−Vd2−Vₘ₁) (14)

The system advantageously enables calculation of the value of contact impedances associated with patient monitoring device including a plurality of electrodes connected to the patient. This includes, for example, an ECG monitoring device that includes any number of electrodes connected to a patient. For example, in a 12-lead ECG system, which contains 10 electrodes, the electrode contact impedances Z1-Z10 would be measured by first considering electrodes Z1-Z3. Using the methods described above, we would calculate values for Z1-Z3. Then we would then consider electrodes Z2-Z4, where three equations and three unknowns could be derived, allowing us to solve for Z4. This would be repeated for each group electrodes Z3-Z10, until all electrode impedances are known. The three electrodes selected for use in calculating respective contact impedances may include electrodes that are all primary electrodes, a combination of primary and secondary electrodes or all secondary electrodes. Thus, the selection of electrodes used to determine the contact impedance may be any electrode so long as each electrode can have a positive or negative current applied thereto enabling pairwise electrical stimulation of the electrodes.

The system advantageously uses three electrodes and three electrode pairs to calculate the contact impedance for each electrode. This enables the control processor (FIG. 1) to rapidly use three linear equations to solve for the respective impedance values rapidly. In another embodiment, additional electrodes and electrode pairs may also be used to determine respective impedance values for electrodes. However, the calculations required in a system that employs more than three electrodes and electrode pairs would be more algebraically complex and require additional processing power. Additionally, the resulting contact impedance calculated using more than three electrode pairs would provide little advantage and would not be any more accurate as compared to the contact impedance calculated using the three electrodes and three electrode pairs.

FIG. 5A depicts a circuit diagram detailing how the impedance on a neutral electrode can be determined. In some ECG monitors, the voltage on the neutral electrode can not be recorded unless it is disconnected from the neutral drive circuitry. In such a monitor, the system described here can be used to measure neutral electrode impedance by disconnecting the neutral driving circuitry and temporarily connecting the electrode to an amplifier so that its voltage can be recorded. Once the neutral electrode is connected to the necessary circuitry for recording, its impedance can be calculated in an identical manner to the V-lead electrodes discussed above. As shown herein, Electrode E is configured to be the neutral electrode. A neutral drive circuit 505 is selectively coupled to Electrode E via switch 513. Neutral drive circuit 505 reduce common mode take the average voltage of the three primary electrodes and then input this signal to an inverting amplifier which is selectively coupled to Electrode E. By inverting the signal that is common to all electrodes and injecting it back into the body through Electrode E, the noise levels experience by the ECG monitor is dramatically reduced.

To measure the impedance Z5 of Electrode E, aspects of the process described above with respect to FIG. 5 are repeated. The voltage differential Vm1 associated with the first electrode pair 300 that includes Electrode A and Electrode B is calculated as discussed above. The voltage differential VmRL of a second electrode pair 510 including Electrode A and Electrode E is calculated. However, as Electrode E in this embodiment is identified as the neutral electrode, the, the switch 513 is caused to move from the first closed position to the second open position decoupling the neutral drive circuit 505 from Electrode E. By decoupling the neutral drive circuit 505 from Electrode E, the system simultaneously applies a positive current Ip from current generator 106A to Electrode A and a negative current In is applied to Electrode E from current generator 106E. The voltage difference VmRL between a voltage on Electrode E and the reference voltage VMP (e.g. Wilson Point) via amplifier 503. The voltage Ve may be calculated using Equation 8 described above except that Vd in Equation 8 would be replaced by Ve representing the voltage on Electrode E and Vm4 is replaced by VmRL which represent the voltage differential between the second pair of electrodes 510. The system repeats this process between Electrode B and Electrode E forming electrode pair 512. A similar measurement can be made between Electrode B and Electrode E where positive current is applied to Electrode B and negative current is applied to Electrode E after decoupling the neutral drive circuit 505 from Electrode E. By repeating the above application with electrode pair 512, the values of impedances Z1, Z2, and Z5 can then be derived by modifying Equations 9 and 10 as follows to be Equations 15 and 16, respectively and using Equations 15 and 16 in conjunction with Equation 11 (repeated below for convenience):

Va−Ve1=I(Z1+Z5) (15)

Vb−Ve2=I(Z2+Z5) (16)

Vm1=I(Z1+Z2) (11)

Where Ve1 is Ve (from modified Eq. 8) in the case that current is injected through electrodes A and D, while Ve2 is Ve (from modified Eq 8) in the case that current is injected through electrodes B and D, Vm1 is measured when current is injected through electrodes A and B, Va is measured when current is injected through electrodes A and E, and Vb is valid when current is injected through electrodes B and E. While Vb is not directly measured, it can be obtained easily either by amplifying and recording Vb directly, or using the differential voltages Vm1, Vm2, and Vm3 in combination with the electrode voltage Va. Once Vb is known, the values of Z1, Z2, and Z% can be calculated using the same manner as discussed above with respect to Equations 12-14, where Z4 would be replaced with Z5 in the embodiment shown in FIG. 5A. Thus, the values of Z1, Z2 and Z5 are shown in Equations 17-19, respectively:

Z1=0.5/I*(Vₘ₁+Va−Ve1−Vb+Ve2) (17)

Z2=0.5/I*(Vₘ₁+Vb−Ve2−V1+Ve1) (18)

Z5=0.5/I*(Va−Ve1+Vb−Ve2−Vₘ₁) (19)

In one embodiment, since disconnecting the neutral drive circuit can introduce 60 Hz noise, it may be necessary that the measured voltage on a respective electrode be averaged over some duration of time (e.g. 50-100 msecs) to average out the noise. In another embodiment, the neutral drive circuitry is selectively connected to a different electrode that is not included in the subset of electrode pairs currently being used to determine electrode impedance while the original neutral electrode impedance is being measured. While the system must wait a predetermined amount of time for the circuit to stabilize once the neutral drive circuitry has been connected to a different electrode, this approach is advantageous in that there is no need to average the recorded voltages because the neutral drive circuitry will attenuate 60 Hz noise.

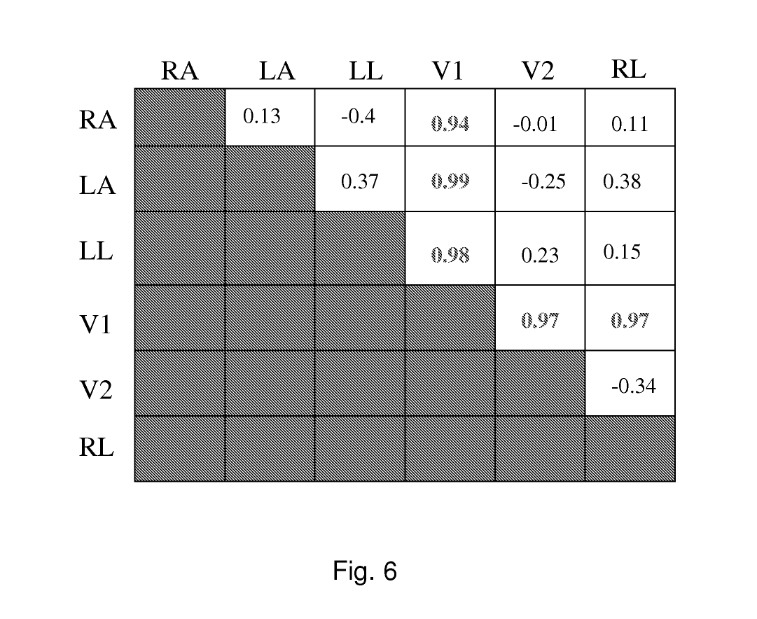

The system described above with respect to FIGS. 1-5A advantageously determines contact impedances for each electrode connected to a patient. If the resulting contact impedance value for a given electrode exceeds a threshold impedance level (e.g. 1GΩ or even infinite impedance due to disconnection of electrode from the patient), the system identifies this contact impedance as invalid. Any pair-wise stimulation applied to an electrode pair that includes an electrode wherein the contact impedance has been identified as invalid will cause the voltage measurement to saturate. One skilled in the art understands that the actual saturation limit will be based on the specific amplifiers, power supplies, etc. In order to determine whether an electrode has an impedance that is high enough to cause saturation and thus be identified as invalid, the system detaches the neutral drive circuitry and pair-wise stimulation is applied between all possible combinations of electrodes to measure the resulting voltage fluctuations between the various electrode pairs. The measured voltages for all electrode pair combinations are entered into a table. The pair-wise voltage values are compared to a saturation level and if the voltage for a particular pair-wise combination at least one of approaches, meets, or exceeds the voltage corresponding to a saturation level, the system automatically avoids measuring the contact impedance for the particular electrodes in the voltage pair. Additionally, the system may notify a healthcare practitioner of an impedance that exceeds a threshold to allow the healthcare practitioner to change or otherwise adjust the electrodes in the electrode pair that are identified as invalid. An example of such a table is shown in FIG. 6, which includes voltage values for all electrode pairs of a 6-electrode system. In this example, electrode V1 has very high impedance which can be inferred from FIG. 6 because the measured voltage is near the saturation level in all cases (the saturation level is set as 1V in this example). Therefore, the impedance of electrode V1 can not be measured using the algorithms described above with respect to FIGS. 1-5A. However, the table indicates that all other electrodes are not saturated, and thus the above method can be used to calculate the impedance of the remaining electrodes. If the electrode being used as the neutral is found to be saturated, then another electrode should be designated as the neutral. In one embodiment, the system automatically performs this pair-wise saturation check prior to determining the contact impedances for the electrodes connected to the system.

If the neutral drive system is detached while the measurements for this table are made, then there will be noise in the voltage measurements, perhaps necessitating the averaging of data over tens or hundreds of milliseconds for each data point in the table. In another embodiment of this system, the neutral drive system is attached during population of the table. In this case, any pairwise stimulation involving the neutral electrode will be performed while the neutral drive system is temporarily detached from the neutral electrode and attached to another electrode that is not currently being stimulated thereby attenuating any common mode noise generated by the patient.

A further issue may arise using the pair-wise voltage data in FIG. 6. In one embodiment, if any of the primary electrodes (right-arm, left-arm, left-leg) have an impedance that is high enough to cause saturation, then this will cause the reference voltage as shown in FIGS. 5 and 5A for calculating contact impedances of the V-leads to be invalid. Since the V-leads are measured relative to the reference voltage, then an invalid reference voltage means all of the V-lead voltages are also invalid. If the system determines that one or more primary electrode is found to be saturated, the system automatically adjusts the manner in which the reference voltage is determined and only average the primary electrodes having voltages that are not saturated or approaching saturation (e.g. within a predetermined numerical distance from the saturation level). The recalculated reference voltage based on the valid primary electrode voltages is used to repeat pair-wise electrical stimulation and measure voltages for all electrode pairs to repopulate the Table shown in FIG. 6.

In summary, the table created in FIG. 6 will allow the system to determine two parameters that may be set before the impedance is measured: (1) determine which electrode to connect to the neutral drive system, and (2) determine which electrodes to use for the calculation of the Wilson point. As an example of how such a table is populated, consider an ECG monitoring system having 6 electrodes connected to a patient. The neutral electrode is detached and the table of voltages is measured (as in FIG. 6). If the table shows that the left-arm electrode voltage is saturated and all other electrodes are not saturated, this indicates that the choice of neutral electrode does not need to be changed, but the Wilson voltage is invalid because it was calculated using the left-arm electrode voltage. Since the Wilson voltage is invalid, all V-lead voltages are invalid. In order to account for this, the system automatically adjusts the manner in which the Wilson voltage is calculated by excluding the left-arm voltage. Thus, in this example, the Wilson voltage would be re-defined to be the average of the left-leg and right-arm (with the left-arm being excluded). The table is then re-measured using the modified Wilson voltage and the table in FIG. 6 is repopulated with valid voltage levels that are not saturated and which are accurate because the Wilson voltage is accurate.

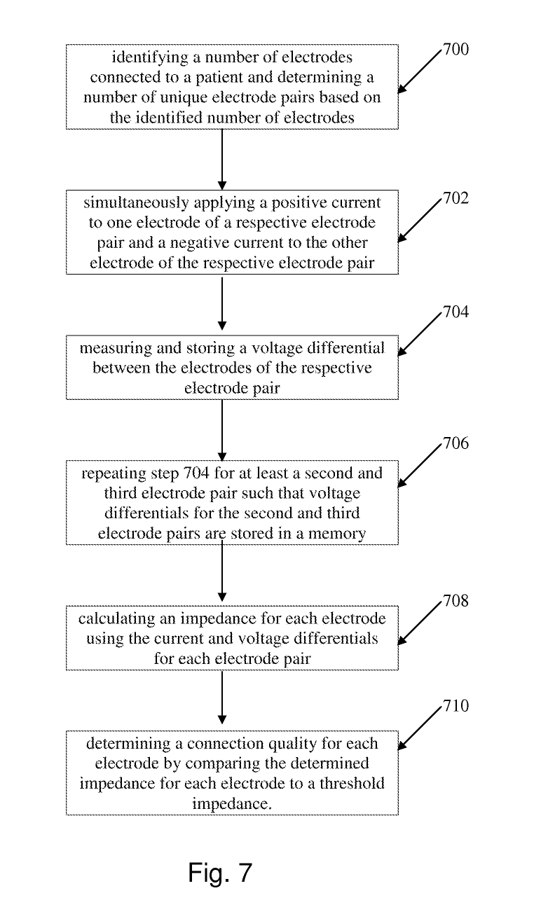

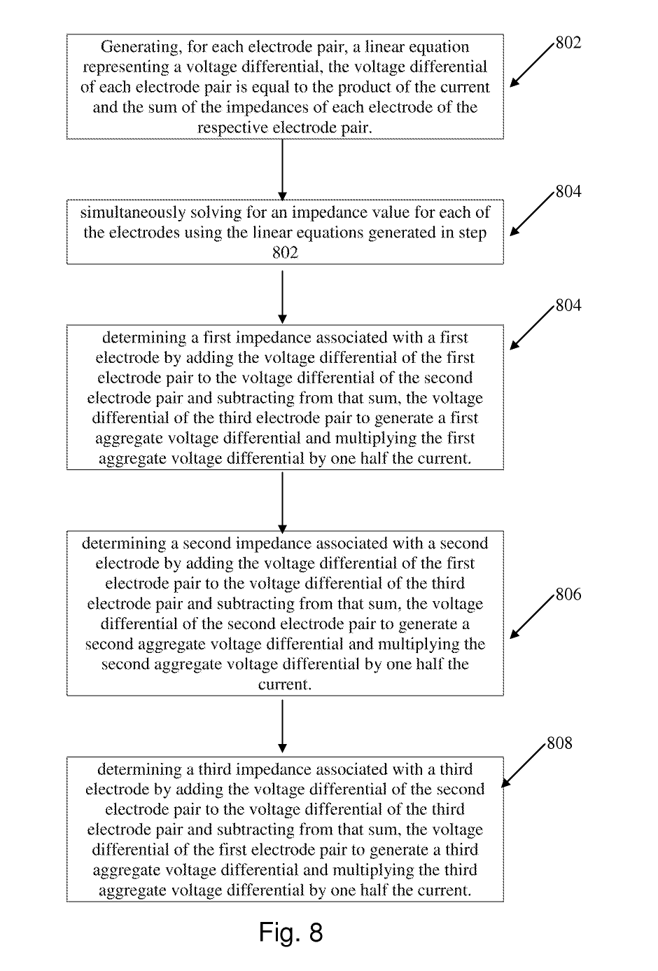

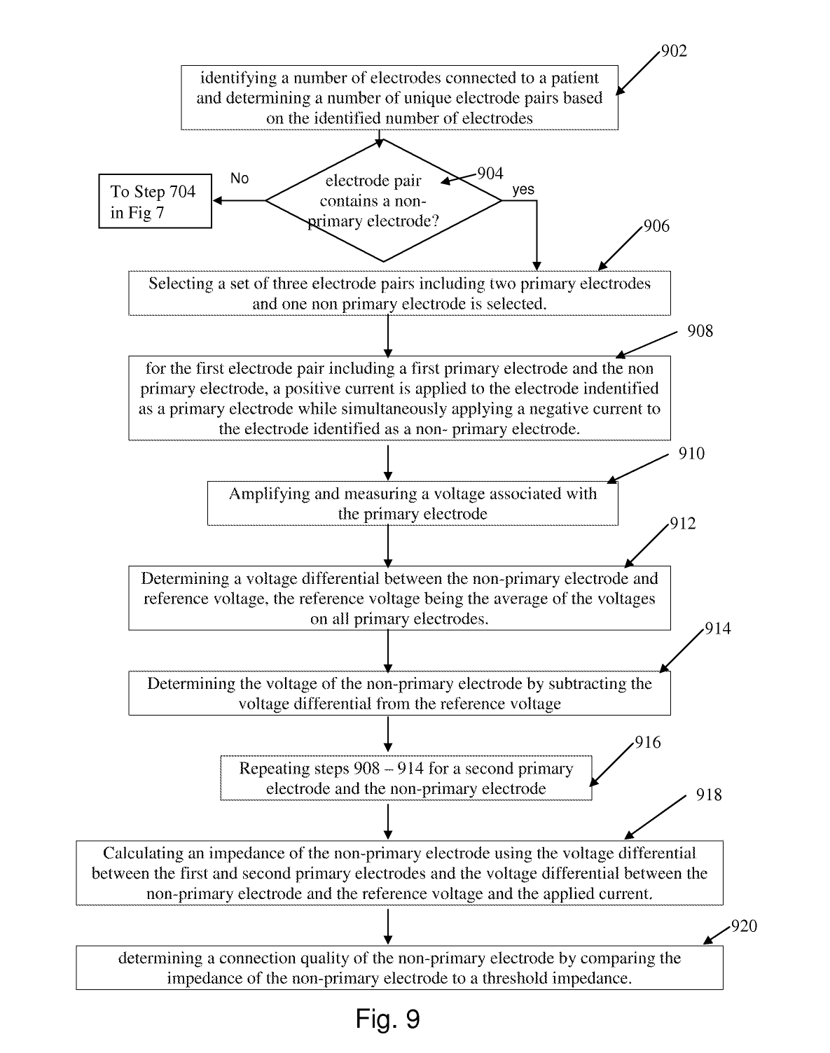

FIG. 7 is flow diagram that details the operation of the system for measuring connection quality of an electrode to a patient. In step 700, a control processor identifies a number of electrodes connected to a patient and determines a number of unique electrode pairs based on the identified number of electrodes. In step 702, a positive current is applied to one electrode of a respective electrode pair simultaneous with a negative current being applied to the other electrode of the respective electrode pair. The positive and negative currents applied in step 702 have equal magnitudes. In step 704, a voltage differential between the electrodes of the respective electrode pair is measured and stored in a memory. In step 706, the activity of step 704 is repeated for at least a second and third electrode pair such that voltage differentials for the second and third electrode pairs are stored in a memory. In step 708, an impedance for each electrode is calculated using the current and voltage differentials for each electrode pair and a connection quality for each electrode is determined by comparing the determined impedance for each electrode to a threshold impedance.

FIG. 8 is a flow diagram representing an algorithm implemented by a control processor for calculating an impedance for respective ones of a plurality of electrodes. The algorithm in FIG. 8 describes the activities performed in step 708 in FIG. 7 and will be described using an example where there are three electrode pairs identified by the control processor. This is described for purposes of example only and one skilled in the art will understand how the follow principles can be scaled up for use in a system that has more than three electrode pairs.

In step 802, for each electrode pair, a linear equation representing a voltage differential thereof is generated. The voltage differential of each electrode pair is equal to the product of the current and the sum of the impedances of each electrode of the respective electrode pair. The control processor simultaneously solves for an impedance values for each of the electrodes using the linear equations generated in step 802. In step 804, a first impedance associated with a first electrode is determined by adding the voltage differential of the first electrode pair to the voltage differential of the second electrode pair and subtracting from that sum, the voltage differential of the third electrode pair to generate a first aggregate voltage differential and multiplying the first aggregate voltage differential by one half the current. In step 806, a second impedance associated with a second electrode is determined by adding the voltage differential of the first electrode pair to the voltage differential of the third electrode pair and subtracting from that sum, the voltage differential of the second electrode pair to generate a second aggregate voltage differential and multiplying the second aggregate voltage differential by one half the current. In step 808, a third impedance associated with a third electrode is determined by adding the voltage differential of the second electrode pair to the voltage differential of the third electrode pair and subtracting from that sum, the voltage differential of the first electrode pair to generate a third aggregate voltage differential and multiplying the third aggregate voltage differential by one half the current.

FIG. 9 is a flow diagram detailing the manner in which contact impedance for both primary and secondary electrodes may be calculated. In step 902, a control processor identifies a number of electrodes connected to a patient and determines a number of unique electrode pairs based on the identified number of electrodes. In step 904, the control processor queries whether or not the electrode pair contains a secondary electrode. If the result of the query in step 904 is negative, then system reverts back to step 704 in FIG. 7. If the result of the query in step 904 is positive, then in step 906, a set of three electrode pairs including two primary electrodes and one secondary electrode is selected. In step 908, for the first electrode pair including a first primary electrode and the secondary electrode, a positive current is applied to the electrode indentified as a primary electrode while simultaneously applying a negative current to the electrode identified as a secondary electrode. In step 910, a voltage associated with the primary electrode is amplified and measured. In step 912, a voltage differential between the secondary electrode and reference voltage is determined, the reference voltage being the average of the voltages on all primary electrodes. The voltage of the secondary electrode is determined by subtracting the voltage differential from the reference voltage in step 914. In step 916, steps 908-914 are repeated for a second primary electrode and the secondary electrode. In step 918, an impedance of the secondary electrode is calculated using the voltage differential between the first and second primary electrodes and the voltage differential between the secondary electrode and the reference voltage and the applied current. In step 920, a connection quality of the secondary electrode is determined by comparing the impedance of the secondary electrode to a threshold impedance.

The connection quality measurement system described above with respect to FIG. 1-9 advantageously enables a connection quality for each electrode connected to a patient to be determined rapidly so as to minimize the time in which the patient monitoring is disrupted. By advantageously identifying electrode pairs and simultaneously applying currents of opposing polarities to each electrodes that make up the electrode pair, the system is able to generate linear equations representing the voltage difference between the electrodes as a product of the current and the sum of the individual impedances of the electrodes in the electrode pair. When a linear equation representing three unique electrode pairs, the system advantageously uses the three linear equations to derive respective impedance values for each electrode connected to the patient. This advantageously enables contact impedances for each electrode to be determined rapidly (<1 sec) because the minimal processing power needed to generate and solve the linear equations. This results in healthcare professionals being provided with more accurate data characterizing the connection quality of electrodes to a patient. The system further advantageously performs a check to determine if the voltage on any particular electrode is approaching saturation. This is particularly important in the realm of ECG monitoring whereby the impedance calculation for a V-leads requires the use of a reference voltage that is the average voltage on all primary electrodes. Thus, the system advantageously excludes electrodes that are determined to be saturated from any impedance calculation thereby providing more reliable indication of connection quality of each particular electrode. This advantageously provides a user with information regarding the connection at each electrode and allows a user to improve monitoring configurations to take into account and utilize electrodes to derive leads that have a higher quality connection. This further enables a user to remedy a degraded connection to improve the quality of the patient data being monitored.

In another embodiment of this system, the ECG is continuously monitored during the measurement of impedance. Turning on and off the current sources during the impedance measurement will produce an artifact in the ECG signal. While such artifacts have the potential to obscure the activity of interest, it is possible to attenuate or remove the artifacts using additional filtering stages, thereby allowing the ECG to be monitored even during the measurement of impedance. This advantageously enables to check the impedance of the various electrodes while simultaneously allowing the patient monitoring device to provide a reduced level of patient monitoring based on the monitored physiological signals. In the embodiment where the monitoring device is an ECG monitor, reduced monitoring may include determining the presence and/or absence of a heartbeat. The application of the above system may also be used in other devices such as exercise equipment or remote monitoring system to determine of the person to which the system is connected is alive.

Although the invention has been described in terms of exemplary embodiments, it is not limited thereto. Rather, the appended claims should be construed broadly to include other variants and embodiments of the invention which may be made by those skilled in the art without departing from the scope and range of equivalents of the invention. This disclosure is intended to cover any adaptations or variations of the embodiments discussed herein.

What is claimed is:

1. An apparatus that determines a quality of a connection of an electrode to a patient, the apparatus comprising:
at least three electrodes selectively connected to a patient for sensing an electrophysiological signal representing a patient parameter
a current source connected to each of the at least three electrodes, the current source able to apply both a positive current and a negative current ;
a control processor connected to the current source and the at least three electrodes, the control processor identifies a number of unique electrode pairs of the at least three electrodes and controls the current source to simultaneously apply a positive current to one electrode and a negative current to an other electrode of each identified electrode pair to determine a connection quality for at least one of the at least three electrodes.

2. The apparatus as recited in claim 1, further comprising
at least three amplifiers, each amplifier connected to receive a voltage of the electrodes in a respective electrode pair to determine a voltage difference associated with the electrode pair; and
the control processor calculates a contact impedance for each electrode of the at least three electrodes based on the determined voltage difference associated with each electrode pair and current applied by said current source.

3. The apparatus as recited in claim 2, wherein
said control processor generates a linear equation associated with each electrode pair, each linear equation equating a voltage difference associated with the respective electrode pair to a product of the positive current applied by the current source and a sum of the impedances of each electrode of the electrode pair.

4. The apparatus as recited in claim 3, wherein
the control processor calculates the impedance of each of the at least three electrodes using the generated linear equations.

5. The apparatus as recited in claim 4, wherein
the control processor simultaneously determines the impedance of each electrode of the at least three electrodes from the determined voltage differences of each electrode pair and the positive current applied by the current source

6. The apparatus as recited in claim 5,
the control processor compares the determined impedance of each electrode with a threshold impedance to determine connection quality for each electrode.

7. The apparatus as recited in claim 6, wherein
the control processor determines a connection quality for an electrode is good when the determined impedance of the electrode is less than a threshold value and a connection quality for an electrode is poor when the contact impedance of the electrode is greater than the threshold value.

8. The apparatus as recited in claim 1, wherein
the at least three electrodes includes a set of primary electrodes and a set of secondary electrodes ; and
said control processor controls the current source to simultaneously apply a positive current to a respective one of the electrodes from the set of primary electrodes and a negative current to a respective one of the electrodes from the set of secondary electrode and determines a voltage difference between the respective one of the secondary electrodes and a reference voltage.

9. The apparatus as recited in claim 1, wherein
the control processor determines an electrode is saturated if the determined voltage difference between the electrode and each electrode paired with the electrode approaches a predetermined value.

10. The apparatus as recited in claim 9, wherein
the control processor automatically excludes an electrode determined to be saturated when determining connection quality of an other electrode.

11. The apparatus as recited in claim 1, wherein
said apparatus is an electrocardiogram monitor.

12. The apparatus as recited in claim 1, further comprising
at least one of (a) an alarm that notifies a user of a determined connection quality data; (b) a display that displays an indicator representing connection quality of at least one electrode to a user; and (c) a communication processor that selectively communicates data representing connection quality to a remote system.

13. An ECG monitoring apparatus that determines a connection quality of an electrode connected to a patient, the apparatus comprising:
a plurality of electrodes coupled to a patient, each of said plurality of electrodes sensing electrical impulses representing at least one patient parameter from the patient;
a current source selectively connectable to the plurality of electrodes that selectively applies one of a positive current and negative current to any of the plurality of electrodes;
a control processor connected to the current source and the plurality of electrodes, the control processor identifies a number of unique electrode pairs from the plurality of electrodes and, for each identified electrode pair, controls the current source to apply a positive current one of the electrodes and a negative current to the other of the electrodes in the electrode pair to generate linear equations representing the voltage difference for the electrode pair and determines connection quality data for respective ones of the plurality of electrodes.

14. The ECG monitoring apparatus of claim 13, further comprises
a plurality of amplifiers, each amplifier connected to receive the voltage of the electrodes in a respective electrode pair to determine the voltage difference associated with the electrode pair.

15. A method of determining a connection quality of an electrode connected to a patient comprising the activities of
providing at least three electrodes that sense an electrophysiological signal representing a patient parameter to a control processor;
connecting a current source able to apply both a positive current and a negative current to each of the at least three electrodes;
identifying a number of unique electrode pairs of the at least three electrodes;
controlling the current source to simultaneously apply a positive current to one electrode and a negative current to an other electrode of each identified electrode pair; and
determining a connection quality for at least one of the at least three electrodes.

16. The method as recited in claim 15, further comprising the activities of
connecting an amplifier to receive a voltage of the electrodes in a respective electrode pair to determine a voltage difference associated with the electrode pair; and
calculating a contact impedance for each electrode of the at least three electrodes based on the determined voltage difference associated with each electrode pair and current applied by said current source.

17. The method as recited in claim 16, further comprising
generating, by the control processor, a linear equation associated with each electrode pair, each linear equation equating voltage difference associated with the respective electrode pair to a product of the positive current applied by the current source Old a sum of the contact impedances of each electrode of the electrode pair.

18. The method as recited in claim 17, further comprising the activity of
calculating the contact impedance of each of the at least three electrodes using the generated linear equations.

19. The method as recited in claim 18, further comprising the activity of
simultaneously determining the contact impedance of each electrode of the at least three electrodes from the determined voltage differences of each electrode pair and the positive current applied by the current source

20. The method as recited in claim 19, wherein the activity of determining connection quality further includes
comparing, by the control processor, the determined contact impedance of each electrode with a threshold contact impedance.

21. The method as recited in claim 20, wherein the activity of determining a connection quality further includes
determining a connection quality for an electrode is good when the determined contact impedance of the electrode is less than a threshold value; and
determining a connection quality for an electrode is poor when the contact impedance of the electrode is greater than the threshold value.

22. The method as recited in claim 15, wherein
the at least three electrodes includes a set of primary electrodes and a set of secondary electrodes; and further comprising the activity of
controlling the current source to simultaneously apply a positive current to a respective one of the electrodes from the set of primary electrodes and a negative current to a respective one of the electrodes from the set of secondary electrode; and
determining a voltage difference between the respective one of the secondary electrodes and a reference voltage.

23. The method as recited in claim 15, further comprising the activity of
determining, by the control processor, an electrode is saturated if when determined voltage difference between the electrode and each electrode paired with the electrode approaches a predetermined value.

24. The method as recited in claim 23, further comprising the activity of
automatically excluding an electrode determined to be saturated when determining connection quality of an other electrode.

25. The method as recited in claim 15, wherein
the method is performed by an electrocardiogram monitor.

26. The method as recited in claim 15, further comprising at least one of the following activities:
(a) notifying a user of a determined connection quality data via an alarm;
(b) displaying an indicator representing connection quality of at least one electrode to a user on a display device; and
(c) selectively communicating data representing connection quality to a remote system via a communication processor.
